(19) United States
(12) Patent Application Publication
(10) Pub. No.: US 2007/0055192 A1
(43) Pub. Date: Mar. 8, 2007

(54) Botulinum toxin in treatment of clubfoot

(75) Inventor: Christine M. Alvarez, Vancouver (CA)

Correspondence Address: Stephen Donovan;Allergan, Inc., 2525 Dupont Drive, Irvine, CA, 92612, US

(73) Assignee: ALLERGAN, INC., Irvine, CA (US)

(21) Appl. No.: 11/504,199

(22) Filed: Aug. 15, 2006

Related U.S. Application Data
(60) Provisional application No. 60/709,430, filed on Aug. 19, 2005.

(51) Int. Cl. A61F 5/00

(52) U.S. Cl. 602/29

(57) ABSTRACT

Botulinum toxin, or other neuromuscular paralytic agents, injected into the lower leg muscle of infants, less than a year old, with idiopathic clubfoot is shown to be an effective therapy in correcting this physical deformity. Following a protocol of manipulations, castings, and injections, clubfoot is effectively treated, and surgical treatment procedures can be avoided.

### CROSS REFERENCE

This application is a nonprovisional utility application which claims priority to related provisional application No. 60/709,430, filed Aug. 19, 2005, the entire content of which application is incorporated herein by reference.

### BACKGROUND OF THE INVENTION

1. Field of Invention

The present invention relates to the field of clubfoot therapy.

2. Description of Related Art

Clubfoot, though predominantly a condition that occurs in isolation (idiopathic clubfoot), is also well-recognized in a variety of pre-existing conditions. Throughout most of the 20th century the mainstay of treatment of clubfoot has been surgical correction involving a variety of techniques (Carroll N C, McMurtry R, Leete S F. The pathoanatomy of congenital clubfoot. Orthop Clin North Am. 1978;9:225-232; Carroll N C. Surgical technique for talipes equinovarus. Oper Tech Orthop. 1993;3:115-120; Crawford A H, Marxen J L, Osterfeld D L. The Cincinnati incision; a comprehensive approach for surgical procedures of the foot and ankle in childhood. J Bone Joint Surg [Am]. 1982;64:1355-1358; McKay D W. New concept of and approach to clubfoot treatment: section II. Correction of the clubfoot. J Pediatr Orthop. 1983;3:10-21; Turco V J. Surgical correction of the resistant clubfoot: one-stage posteromedial release with internal fixation. A preliminary report. J Bone Joint Surg [Am]. 1971;53:477-497). “Good” to “excellent” initial results have been reported in the range of 52% to 91% for these surgical methods (Herzenberg J E, Radler C R, Bor N. Ponseti versus traditional methods of casting for idiopathic clubfeet. J Pediatr Orthop. 2002;22:517-521; Roye D P, Roye B D. Idiopathic congenital talipes equinovarus. J Am Acad Orthop Surg. 2002;10:239-248). However, surgical interventions have associated reported complications in 11% to 50% of cases (Applington J P, Riddle C D. Avascular necrosis of the body of the talus after combined medial and lateral release of congenital clubfoot. South Med J. 1976;69:1037-1038; Atar D, Lehman W B, Grant A D. Complications in clubfoot surgery. Orthop Rev. 1991;20:233-239; Miller J H, Bernstein S M. The roentogenographic appearance of the corrected clubfoot. Foot Ankle. 1986;6:177-183; Schlafly B, Butler J E, Siff S J. The appearance of the tarsal navicular after posteromedial release for clubfoot. Foot Ankle. 1985;5:222-237). Often, complications are related to the Achilles tendon that may result in calcaneal deformity, from over-lengthening of the tendon or equinus from insufficient posterior release with or without under-lengthening of the Achilles tendon (Crawford A H, Gupta A K. Clubfoot controversies: complications and causes for failure. AAOS Instr Course Lect. 1996;45:339-346). Postoperative gait analysis has shown abnormalities in ankle rocker formation and timing (Alkjaer T, Pedersen E N, Simonsen E B. Evaluation of the walking pattern in clubfoot patients who received early intensive treatment. J Pediatr Orthop. 2000;20:642-647; Asperheim M S, Moore N, Carroll N C, et al. Evaluation of residual clubfoot deformities using gait analysis. J Pediatr Orthop B. 1995;4:49-54; Hee H T, Lee E H, Lee G S. Gait and pedographic patterns of surgically treated clubfeet. J Foot Ankle Surg. 2001;40:287-294; Karol L A, Concha M C, Johnston C E 2d. Gait analysis and muscle strength in children with surgically treated clubfeet. J Pediatr Orthop. 1997;6:790-795; Karol L A, Mayberry S, O'Brien 0, et al. Gait in patients with clubfeet: a comparison of physical therapy versus surgical release. Pediatric Orthopaedic Society of North America 2003 Annual Meeting, Amelia Island, Fla., May 2-4, 2003; Kuo K N, Hennigan S P, Hastings M E. Long term results of clubfoot release, outcome study and gait analysis. Pediatric Orthopaedic Society of North America 2003 Annual Meeting, Amelia Island, Fla., May 2-4, 2003; Otis J C, Bohne W H. Gait analysis in surgically treated clubfoot. J Pediatr Orthop. 1986;6:162-164; Widhe T, Berggren I. Gait analysis and dynamic foot pressure in the assessment of the treated clubfoot. Foot Ankle Int. 1994;15:186-190) and kinetic studies have revealed a significant decrease in power generation, with an average triceps surae muscle complex strength reduction of 27% after one Achilles tendon lengthening (Karol L A, Concha M C, Johnston C E 2d. Gait analysis and muscle strength in children with surgically treated clubfeet. J Pediatr Orthop. 1997;6:790-795; Karol L A, Mayberry S, O'Brien O, et al. Gait in patients with clubfeet: a comparison of physical therapy versus surgical release. Pediatric Orthopaedic Society of North America 2003 Annual Meeting, Amelia Island, Fla., May 2-4, 2003; Kuo K N, Hennigan S P, Hastings M E. Long term results of clubfoot release, outcome study and gait analysis. Pediatric Orthopaedic Society of North America 2003 Annual Meeting, Amelia Island, Fla., May 2-4, 2003; Widhe T, Berggren I. Gait analysis and dynamic foot pressure in the assessment of the treated clubfoot. Foot Ankle Int. 1994;15:186-190).

Ponseti published a protocol of serial manipulations and castings in 1980, reporting, at that time, that traditional surgery could be avoided in 89% of cases (Laaveg S J, Ponseti I V. Long-term results of treatment of congenital clubfoot. J Bone Joint Surg [Am]. 1980;62:23-31) and, more recently, excellent functional and clinical outcomes in 78% of patients at 30-year follow-up (Cooper D M, Dietz F R. Treatment of idiopathic clubfeet: a thirty-year follow-up note. J Bone Joint Surg [Am]. 1995;77:1477-1489). Unresolved hindfoot equinus, occurring in 70% to 90% of cases, was treated with percutaneous Achilles tenotomy. Other investigators have reported their experiences using the method described by Ponseti, quoting similarly favorable outcomes, albeit over a shorter follow-up (<3 years). Similarly, the physical therapy method also requires a significant rate of Achilles tenotomy (Bensahel H, Guillaume A, Desgrippes Y. Results of physical therapy for idiopathic clubfoot: a long-term follow-up. J Pediatr Orthop. 1990;10:189-192). Bensahel et al reported a 26% surgical rate, which includes Achilles tenotomy (Bensahel H, Guillaume A, Desgrippes Y. Results of physical therapy for idiopathic clubfoot: a long-term follow-up. J Pediatr Orthop. 1990;10:189-192) and Richards and Wilson (Richards B S, Wilson H. Non-operative clubfoot treatment using physical therapy. International Society of Orthopaedic Surgery and Traumatology 3rd International Clubfoot Congress, San Diego, Calif., August 2002) reported a 47% overall tenotomy rate.

A method of clubfoot therapy that permits restoration of the normal foot position without requiring surgical intervention is desirable and needed. Novel pharmaceutical compositions comprising botulinum toxin or toxins are derived from the bacterium Clostridium botulinum and cause reversible muscle denervation by blocking the release of acetylcholine at the neuromuscular junction, leading to muscle relaxation. Botulinum toxin, specifically botulinum toxin A, is currently used in the treatment of cerebral palsy, poststroke spasticity, and other instances of inappropriate muscle contraction.

Reiter (Reiter, F, Danni, M., Lagalla, G., Ceravolo, G., Provinciali, L. 1998. Low dose botulinum toxin eith ankle taping for the treatment of spastic equinovarus foot after stroke. Arch Phys Med Rehabil 79:532-535) teaches use of BTX-A in combination with taping methods for treatment of spasticity following stroke. This treatment was administered to adult patients with previously normal foot function.

Delgado (Delgado et al. A preliminary report of the use of botulinum toxin type A in infants with clubfoot: four case studies. Journal of Pediatric Orthopedics. 2000;20(4):533-8) reported application of a nonsurgical intervention for clubfoot therapy with the initial management involving physical therapy. A group of infant patients under the age of 1 year with clubfoot deformity were treated with BTX-A to resolve the abnormal foot posture. Several of these patients' conditions were dystonic in nature due to underlying disorders, and could not be considered ‘idiopathic’ in nature. Delgado's methods involved injection into both the gastrocnemius and the posterior tibial muscles, and dosages varied from 6-11 IU/kg. Delgado's methods used multiple muscle sites at multiple irregular intervals (on average three separate injection events) for all patients. In addition, 50% of the patients required additional surgery after 1 year of age.

Cummings (53) presented a study suggesting that use of Botox™ in combination with the Ponseti methods was not a successful treatment for clubfoot.

There are a wide variety of approaches to therapy of spastic muscle disorders, particularly clubfoot in infants. Given the range of casting and manipulation methods, and the range in patient classification, dosages and compositions of botulinum toxin used, the method may have promise, however very specific diagnoses and treatment methodologies may be required. The conclusions of Cummings (Cummings, R J and Shanks, D E. A prospective randomized double-blind study of the usefulness of botox as an adjunct to serial manipiulation and casting for congenital clubfeet. Pediatric Orthopaedic Society of North America Annual Meeting. Ottawa, Canada May 12-15, 2005) suggest that the details of the methods used, including the injected sites, matter significantly. Merely ‘mashing together’ various treatment regimens as may be suggested by some studies will not be successful in both primary treatment of idiopathic clubfoot and prevention of relapse.

If surgical methods can be avoided, scarring and surgical complications are also avoided, and in severe cases, multiple correction attempts may be made. Surgical correction of clubfoot is limited—tenotomy cannot be performed more than twice without causing structural weakness in the tendon having adverse effects on recovery and gait. Non-invasive methods are better accepted by parents and caregivers, and are less distressing on the patient.

### SUMMARY OF THE INVENTION

In accordance with one aspect of the invention, there is provided a method of treating clubfoot in a patient, the method comprising administering manipulation and casting therapy to a clubfoot affected foot of a patient until hindfoot stall is achieved, administering a medicament comprising a neuromuscular paralytic agent to a triceps surae muscle complex adjoining the clubfoot-affected foot, and, applying a brace to the clubfoot-affected foot, wherein the clubfoot-affected foot is held in a normal posture.

In accordance with another aspect of the invention, there is provided a method of diagnosing hindfoot stall in a patient receiving treatment for clubfoot (have moved the definition of hindfoot stall to the ‘Detailed Description’), the method comprising acquiring a first Pirani score for a clubfoot-affected foot of a patient, acquiring a second Pirani score for the clubfoot-affected foot of a patient at a later time, comparing the second Pirani score with the first Pirani score, and determining a magnitude of change, wherein the magnitude of change less than a specified cut-off value indicates the presence of said hindfoot stall in a clubfoot-affected foot of the patient.

A cutoff value below which a hindfoot stall may be diagnosed may comprise an ankle dorsiflexion range from about −5 degrees to about +5 degrees

In accordance with another aspect of the invention, there is provided a method of treating hindfoot stall in a patient receiving treatment for clubfoot, the method comprising administering to a clubfoot-affected foot of a patient diagnosed with hindfoot stall a medicament comprising a neuromuscular paralytic agent to a triceps surae muscle complex adjoining the clubfoot-affected foot, and applying a brace to the clubfoot-affected foot, wherein the clubfoot-affected foot is held in a normal posture.

The manipulation and casting therapy may comprise the Ponseti methodology, or may employ a physical therapy method and manipulation method known in the art, for example, the Kite method.

The neuromuscular paralytic agent may comprise a medicament comprising a botulinum toxin type A or type B, or other botulinum toxin-comprising medicaments.

A suitable brace system may comprise a Denis-Browne boot and brace or other corrective boot and brace systems or orthoses, such as knee-ankle-foot orthoses.

Normal foot posture may comprise a hindfoot that is neutral to valgus and plantigrade, a midfoot which is neutral with mild limits of supination and pronation, and a forefoot that is neutral with the heel bisector at the ⅔ space plus or minus 1 heel bisector.

Clubfoot may be idiopathic or non-idiopathic in nature. Clubfoot may occur in one or both feet of an individual. Both feet may be treated simultaneously or in sequence. Non-idiopathic clubfoot refers to clubfoot occurring in an individual in the presence of a coexisting disorder. Such coexisting disorders may include myelomenginocoele, arthrogryposis, migration abnormalities of the brain, cerebral palsy, positional deformity, neurological disorders, spina bifida or other unspecified genetic syndromes resulting in the presence of clubfoot in a patient. Idiopathic clubfoot is clubfoot occurring in an individual in the absence of coexisting disorders.

Other aspects and features of the present invention will become apparent to those ordinarily skilled in the art upon review of the following description of specific embodiments of the invention in conjunction with the accompanying figures.

### BRIEF DESCRIPTION OF THE DRAWINGS

In drawings which illustrate embodiments of the invention,

FIG. 1 is a graphical illustration of the posterior view of the lower leg, showing the BTX-A injection pattern.

FIG. 2 is a flow chart depicting the selection and treatments of the trial patients.

FIG. 3 shows superimposed graphs representing outcome scores for group 1 patients. Bar graphs show dorsiflexion scores for both the knee in flexion (DFF) and in extension (DFE) and line graphs below show Pirani scores. Treatment timeline from initial visit to 27 months after BTX-A injection is shown on the horizontal axis.

FIG. 4 shows superimposed graphs representing outcome scores for group 2 patients. Bar graphs show dorsiflexion scores for both the knee in flexion (DFF) and in extension (DFE) and line graphs below show Pirani scores. Treatment timeline from initial visit to 27 months after BTX-A injection is shown on the horizontal axis.

### DETAILED DESCRIPTION

Any terms not directly defined herein shall be understood to have the meanings commonly associated with them as understood within the art of the invention. As employed throughout the specification, the following terms, unless otherwise indicated, shall be understood to have the following meanings.

“Clubfoot” or “clubfoot deformity” as used herein refers to the presence of a foot of a human that cannot be corrected with manipulation to a normal flexible forefoot, midfoot, and hindfoot position. Clubfoot may occur in one or both feet of an individual. Clubfoot may occur as a single isolated defect with no underlying cause, or may occur in conjunction with one or more coexisting disorders.

“Idiopathic clubfoot” or “idiopathic clubfoot deformity” as used herein refers to clubfoot with no coexisting disorders.

“Non-idiopathic clubfoot” or “non-idiopathic clubfoot deformity” as used herein refers to clubfoot in the presence of a coexisting disorder. Such coexisting disorders may include myelomenginocoele, arthrgryposis, migration abnormalities of the brain, cerebral palsy, positional deformitie, neurological disorders, spina bifida, trichorhinophalangeal syndrome or other unspecificed genetic syndromes resulting in the presence of clubfoot in a patient.

“Normal foot posture” or “normal posture of the foot” as used herein refers to a hindfoot that is in neutral to valgus and plantigrade, a midfoot which is neutral with mild limits of supination and pronation, and a forefoot that is neutral with the heel bisector at the ⅔ space plus or minus 1 heel bisector.

“Triceps surae complex”, as used herein, refers collectively to the gastrocnemius and soleus muscles of the lower leg. An alternative term may be gastrosoleus or gastrocsoleus, also referring collectively to the gastrocnemius and soleus muscles of the lower leg.

“Percutaneous Achilles tenotomy”, as used herein, refers to a surgical procedure to lengthen the Achilles tendon, where under sterile conditions, a surgical blade is inserted deep into the Achilles tendon near the insertion into the calcaneus and the tendon incompletely transected. A full tenotomy transects the tendon and releases it, while an Achilles lengthening procedure actually gives length to tendon but reattaches the two ends together, thereby allowing control in the desired extent of lengthening.

A “Pirani score” as used herein refers to a scoring system for assessment of clubfoot (Pirani S, Outerbridge H, Moran M, Sawatsky B J. A method of evaluating the virgin clubfoot with substantial interobserver reliability. Pediatric Orthopaedic Society of North America 1995 Annual Meeting, Miami, Fla., May 1995; Flynn J M et al. An independent assessment of two clubfoot-classification systems. Journal of Pediatric Orthopedics. 18(3):323-7, 1998). The Pirani score used comprised three measures for the midfoot and three for the hindfoot (each scored as 0, 0.5, or 1.0, for a total score ranging from 0 to 6, the higher score reflecting the more severe deformity). An alternate classification schema for clubfoot is that of Dimeglio and Bensahel (“Dimeglio system”) (Dimeglio A, Bensahel H, Souchet Ph, Mazeau P, Bonnet F. Classification of clubfoot. Journal of Pediatric Orthopaedics (Br) 1995;4:129-136). The Dimeglio system characterizes the severity of clubfoot deformity into four grades, based on varus and equinus in the sagittal plane, derotation of the calcaneopedal block, and position of the forefoot relative to the hindfoot in the horizontal plane. A Grade 1 foot is mild (soft-soft); a Grade 2 foot is moderate (soft to stiff); a Grade 3 foot is severe (stiff to soft) and a Grade 4 foot is very severe, pseudoarthrogrypotic feet (stiff-stiff). The efficacy of correction of clubfoot may be assessed by the degree of ankle dorsiflexsion, as assessed with the knee in both extension and flexion.

Clubfoot correction resulting from treatment refers to a response to this treatment. An alternate term is ‘clubfoot management’. The corrected clubfoot deformity is measured by the amount of motion achieved by a patient. This is based on clubfoot treatment decisions, specifically, if the patient is able to fit into corrective bracing (achieve ankle dorsiflexion of 10 degrees or greater) which is an indicator of correction of the clubfoot.

A patient relapse, as used herein refers a loss of dorsiflexion (with knee in flexion <5 degrees and/or with knee in extension <0 degrees), in a patient currently receiving or having previously received therapeutic intervention for clubfoot.

“Equinus”, or “talipes equinus”, as used herein, refers to a deformity of the foot in which the sole is flexed below neutral or in the plantarflexed range (specifically ankle dorsiflexion is less than 0 degrees). Walking is done on the toes without touching the heel to the ground. ‘Toe walking’ is an alternate term to describe this altered foot position's resulting gait.

“Hindfoot stall”, as used herein, refers to a state wherein the forefoot may be abducted to 60 degrees with persistent hindfoot equinus present, or if the lateral radiograph of the foot demonstrates a downgoing calcaneus and/or talocalcaneal parallelism.

An ‘antagonist’, as used herein, refers to a chemical entity that acts to reduce the physiological activity of another chemical entity, for example by combining with and blocking the receptor of the endogenous chemical entity.

A “chemical entity”, as used herein refers to small organic or inorganic molecules with distinct molecular composition made synthetically, found in nature, or of partial synthetic origin. Included in this group are nucleotides, nucleic acids, amino acids, peptides, proteins, or complexes comprising at least one of these entities, such as a chromosome.

A “medicament”, as used herein, refers to a chemical entity capable of producing an effect that may be administered to a patient or test subject. The effect may be chemical, biological or physical, and the patient or test subject may be human, or a non-human animal, such as a rodent or transgenic mouse. The medicament may be comprised of the effective chemical entity alone or in combination with a pharmaceutically acceptable excipient.

A pharmaceutically acceptable excipient includes any and all solvents, dispersion media, coatings, antibacterial, antimicrobial or antifungal agents, isotonic and absorption delaying agents, and the like that are physiologically compatable. The excipient may be suitable for intravenous, intraperitoneal, intramuscular, intrathecal or oral administration. The excipient may include sterile aqueous solutions or dispersions for extemporaneous preparation of sterile injectable solutions or dispersion. Use of such media for preparation of medicaments is known in the art.

A pharmacologically effective amount of a medicament as used herein refers to using an amount of a medicament present in such a concentration to result in a therapeutic level of drug delivered over the term that the drug is used. This may be dependent on mode of delivery, time period of the dosage, age, weight, general health, sex and diet of the subject receiving the medicament.

The medicaments of the present invention may be formulated for administration by any of various routes. The medicaments may include an excipient in combination with the effective chemical entity, and may be in the form of, for example, tablets, capsules, powders, granules, lozenges, pill, suppositories, liquid or gel preparations, or an injectable formulation, suitable for subcutaneous, intramuscular, intravenous, intraperitoneal, intra-arterial or other modes of injectable delivery. Medicaments may be formulated for parenteral administration in a sterile medium. The medicament may be dissolved or suspended in the medium. Medicaments may be formulated for a subdermal implant in the form of a pellet, rod or granule. The implant or implants may be inserted subcutaneously by open surgery or by use of a trochar and cannula under local anaesthesia. The implant may be periodically replaced or removed altogether. Medicaments may also be formulated for transdermal administration using a patch. The patch is applied to a shaven area of the skin of the patient while the medicament is desired for administration, and removed when no longer needed.

A “neuromuscular paralytic agent”, as used herein, refers to an acetylcholine antagonist, an acetylcholine release inhibitor or a cholinergic release inhibitor. Neuromuscular paralytic agents generally exert their effect by blocking acetylcholine release from a presynaptic terminal of a nerve ending at a neuromuscular junction. Administration of a neuromuscular paralytic agent in a medicament may result in a degree of paralysis of the muscle at the site of administration. The paralysis may be reversible or irreversible.

“Botulinum toxin”, as used herein, refers to a neuromuscular paralytic agent normally produced by the Clostridium botulinum bacteria. Botulinum toxin A, botulinum type A toxin or botulinum toxin type A may be abbreviated as BTX-A. Botulinum toxin B, botulinum type B toxin or botulinum toxin type B may be abbreviated as BTX-B. Botox™ (Allergan), also referenced as Oculinum™ or Dysport™, is a commercially produced medicament comprising BTX-A. Myobloc™ or Neurobloc™ (Solstice Neurosciences) is a commercially produced medicament comprising BTX-B. Other botulinum toxins include botulinum toxin C, botulinum toxin D, botulinum toxin E, botulinum toxin F and botulinum toxin G—these may be administered in the form of a medicament comprising such toxins.

Physical therapy approaches for the treatment of clubfoot in infants vary. The most commonly used and peer-reviewed method is that of Bensahel and Dimeglio (Bensahel H, Guillaume A, Desgrippes Y. Results of physical therapy for idiopathic clubfoot: a longterm follow up. Journal of Pediatric Orthopaedics 1990;10:189-192; Seringe R, Atia R. Idiopathic congenital clubfoot. Results of functional treatment. Revue de Chirurgie Orthopedique et Reparatrice de I'Appareil Moteur 1990;76:490-501; Souchet P, Bensahel H, Themar-Noel C, Pennecot G, Csukonyi Z. Functional treatment of clubfoot: A new series of 350 idiopathic clubfeet with long-term follow-up. Journal of Pediatric Orthopaedics (Br) 2004;13:189-196). The Ponseti method involves a set methodology of manipulations and casting of the foot and is an intensive and involved method, requiring significant caregiver compliance and participation. The Ponseti method generally requires that all components of the clubfoot be corrected simultaneously, except for equinus, which is corrected last. The cavus is corrected together with the adduction by supinating and abducting the forefoot in proper alignment with the hindfoot. With the arch well moulded and the foot in slight supiation, the entire foot can be gently and gradually abducted under the talus, which is secured against toration in the ankle mortise by applying counterpressure (with the thumb of the therapist) against the lateral part of the talus head. Heel varus is corrected when the entire foot is fully abducted. Finally, equinus is corrected by performing an Achilles tenotomy in 80% of cases.

A pivotal point in the manipulation and casting approach to clubfoot correction is hindfoot stall—the angle of the foot no longer improves with continuing treatment. It would be optimal to correct the deformity completely without ever encountering hindfoot stall however, this is often not the case. Only 10-20% of clubfoot cases are resolvable without intervention for hindfoot stall. Intervention for hindfoot stall is also refered to as “hindfoot defunctioning”. Dorsiflexing of the foot may be generally facilitated by a simple percutaneous tenotomy of the tendoachillis, and this is also the current approach in the art to hindfoot stall. Percutaneous Achilles tenotomy, while simple to perform technically, may lead to immediate complications causing neurovascular injury. Further, it may lead to future complications of such as skin scarring and deep tissue fibrosis resulting in reduced ankle joint range of motion and power push off, affecting the gait.

It would be ideal to be able to complete the protocol of manipulations and castings or physical therapy on the clubfoot-affected patient without disturbing the integrity of the Achilles tendon or increasing the frequency of visits beyond a weekly basis.

Neuromuscular paralytic agents such as botulinum toxin, for example (BTX-A) are potent neuromuscular blocking agents that cause partial reversible muscle paralysis (Brin M F, ed. Spasticity: etiology, evaluation, management, and the role of botulinum toxin A. Muscle Nerve. 1997;20(Supp 6):61-91; Eames N W, Baker R, Hill N, et al. The effect of botulinum A toxin on gastrocnemius length: magnitude and duration of effect. Dev Med Child Neurol. 1999;41:226-232; Juzans P, Comella J X, Molgo J, et al. Nerve terminal sprouting in botulinum A-treated mouse levator auris longus muscle. Neuromuscul Disord. 1996;6:177-185). BTX-A acts by blocking acetylcholine release from the presynaptic terminal of peripheral nerve endings at the neuromuscular junction. Intramuscular injection of BTX-A has been shown to lead to partial paralysis of the respective muscle and has been shown to be effective in a number of disorders (Bang M S, Chung S G, Kim S B, et al. Change of dynamic gastrocnemius and soleus muscle length after block of spastic muscle in cerebral palsy. Am J Phys Med Rehabil. 2002;81:760-764; Brin M F, ed. Spasticity: etiology, evaluation, management, and the role of botulinum toxin A. Muscle Nerve. 1997;20(Supp 6):61-91; Eames N W, Baker R, Hill N, et al. The effect of botulinum A toxin on gastrocnemius length: magnitude and duration of effect. Dev Med Child Neurol. 1999;41:226-232; Juzans P, Comella J X, Molgo J, et al. Nerve terminal sprouting in botulinum A-treated mouse levator auris longus muscle. Neuromuscul Disord. 1996;6:177-185; Klein A W. Complications and adverse reactions with the use of botulinum toxin. Semin Cutan Med Surg. 2001;20:109-120; Scott A B. Botulinum A injection of eye muscles to correct strabismus. Trans Am Ophthalmol Soc. 1981;79:734-770) and safe across all ages studied (2 days old to adults) (8,17,32). Side effects are rare and transient (Bakheit A M, Severa S, Cosgrove A, et al. Safety profile and efficacy of Botulinum toxin A (Dysport) in children with muscle spasticity. Dev Med Child Neurol. 2001;43:234-238; Juzans P, Comella J X, Molgo J, et al. Nerve terminal sprouting in botulinum A-treated mouse levator auris longus muscle. Neuromuscul Disord. 1996;6:177-185; Klein A W. Complications and adverse reactions with the use of botulinum toxin. Semin Cutan Med Surg. 2001;20:109-120) and repeated doses may be given if necessary without concern of inducing any long-term complications (Brin M F, ed. Spasticity: etiology, evaluation, management, and the role of botulinum toxin A. Muscle Nerve. 1997;20(Supp 6):61-91; Mooney J F, Koman L A, Smith B P. Pharmacologic management of spasticity in cerebral palsy. J Pediatr Orthop. 2003;23:679-686).

Injection of a neuromuscular blocking agent, for example BTX-A, in the triceps surae muscle complex as an adjunct to manipulation and casting avoids the need for Achilles tenotomy as well as the need for daily manipulations while producing comparable long-term results.

A possible mechanism of action of BTX-A is contemplated by the inventors. Regarding the triceps surae muscle complex weakness, consider the length passive force curve as it relates to muscle contraction (Johnson L, ed. Essential Medical Physiology, 2nd ed. Philadelphia: Lippincott-Raven, 1998). In theory, the relationship between the actin and myosin filament overlap is altered by cutting the Achilles tendon allowing an increase in overlap of the filaments as the muscle recoils on itself. This then reduces the degree of excursion the muscle can achieve, thereby reducing power generation. BTX-A thus causes the muscle to relax, resulting in lengthening of the muscle, and decreasing overlap of the myofibrils. However, not all the myofibrils are affected, as only those units exposed to the BTX-A relax; therefore, the effect on the muscle fiber overlap may be attenuated. The BTX-A route may then be a more sound physiologic approach to improving hindfoot range of motion while preserving muscle fiber excursion and ultimately power generation. This method may permits repeated injections without irreversible damage to the triceps surae muscle complex physiology, allowing for preserved functional outcomes.

BTX-A compares favorably to Achilles tendon tenotomy as a method to attenuate the function of the triceps surae muscle complex in patients with both idiopathic and non-idiopathic clubfoot. These results are comparable to those reported in the literature using Ponseti's method or the physical therapy method and were achieved without the need for tenotomy or more frequent manipulations. A key step in the Ponseti method is defunctioning of the gastrocnemius by tenotomy. The method presented herein represents an improvement on the Ponseti method for the treatment of idiopathic and non-idiopathic clubfoot. The use of BTX-A as an adjunctive therapy in the noninvasive approach of manipulation and casting in idiopathic and non-idiopathic clubfoot is an effective and safe alternative, and one that may be preferable to parents. The present invention further provides that BTX-A injection may also be a suitable therapeutic approach in patients with lower leg muscle spasm disorders lacking a neurological basis. These disorders may include, but are not limited to, idiopathic toe walkers and Legge-Calve-Perthes disease, in addition to idiopathic and non-idiopathic clubfoot.

Methods

Patient Recruitment

Infants referred for suspected clubfoot deformity to four of the six orthopaedic surgeons at British Columbia's Children's Hospital were reviewed consecutively for inclusion in the clubfoot deformity studies. Written informed consent was obtained from all parents. The study protocol and procedures were approved by the University of British Columbia Screening Committee for Research Involving Human Subjects.

Patients were excluded from the study of idiopathic clubfoot if other coexisting disorders were identified, or if they had been previously operated on for their clubfoot deformity or were unable to comply with the treatment protocol. Patients identified as non-idiopathic clubfoot were considered in subsequent studies, separate from that of the idiopathic group. Two idiopathic clubfoot groups were identified: group 1 (<1 month of age at initial presentation) and group 2 (1-8 months of age at initial presentation).

Scoring, Assessment and Follow-Up

Pirani scores were recorded upon entry to all studies—idiopathic and non-idiopathic clubfoot. Pirani scores and dorsiflexion of the ankle with the knee in flexion equal to or greater than 90 degrees and then in maximum knee extension was measured in sequence only after hindfoot stall was encountered and at every visit thereafter. The foot was not manipulated prior to these measurements so that the least amount of dorsiflexion was measured. Clinical photographs obtained included the following views: medial, lateral, plantar, and dorsal views of the foot and tibia.

Patient relapses, defined as an increase in the Pirani score above 1.5 or loss of dorsiflexion (with knee in flexion <5 degrees and with knee in extension <0 degrees), were recorded as well as the reason for relapse (compliance with protocol vs. fitting problem).

Patients were seen weekly until boots and bars were initiated. Casts were removed by the caregiver prior to coming into the clinic and were off for a minimum of 5 minutes and a maximum of 12 hours (on average 2 hours). Visits were then monthly until 9 months after BTX-A injection, every 3 months until 3 years, and every 6 months after 3 years of age. If there were any recurrences or complications, visits between these intervals were made and recorded.

Manipulation and Casting

For both idiopathic and non-idopathic clubfoot studies, patients underwent manipulation and castings emulating Ponseti's principles (Ponseti I V. Congenital Clubfoot: Fundamentals of Treatment. New York: Oxford University Press, 1996; Ponseti I V. Common errors in the treatment of congenital clubfoot. Int Orthop. 1997;21:137-141) until hindfoot stall was encountered. The goals of the initial manipulations using the Ponseti methods were first to address the cavus deformity by dorsiflexing the first ray through the application of pressure to the plantar aspect of the head of the first metatarsal to achieve forefoot supination that matched the hindfoot varus. The second goal was to abduct the forefoot once the midfoot became unlocked using counterpressure on the head of the talus. Hindfoot stall was declared if the forefoot could be abducted to 60 degrees but hindfoot equinus persisted (ankle dorsiflexion in knee flexion of 5±5 degrees or in knee extension of 0±5 degrees) or if the lateral radiograph of the foot showed a downgoing calcaneus and/or talocalcaneal parallelism. Pronation of the forefoot or manipulation of the calcaneus was not directly attempted.

Injection Technique

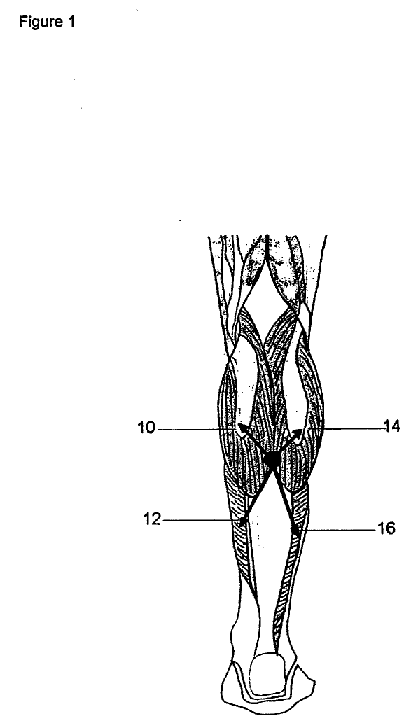

To attenuate the function of the triceps surae muscle complex, BTX-A at 10 IU/kg was injected into this muscle complex at hindfoot stall (FIG. 1). In patients with bilateral clubfeet the total dose was divided equally between the two legs. BTX-A was diluted to 100 IU/1 mL of unpreserved normal saline, giving a concentration of 10 IU/0.1 mL.

Prior to injection, local anesthetic cream was applied to the patient's calf at the junction of the distal and middle third of the triceps surae muscle bulk and covered with an impermeable dressing for 30 minutes. The patient was then placed in a prone position over a pillow. BTX-A was injected from one skin site in a stellate pattern into the triceps surae muscle complex, ensuring that all four quadrants were injected (injection sites are indicated at points 10,12, 14 and 16). The muscle was then massaged for 30 seconds to disperse the drug. The total amount of BTX-A (10 IU/kg) was divided approximately equally between the four injection sites of the leg. If both legs were injected in a patient, the total amount of BTX-A was divided approximately equally among the 8 sites over both legs.

Immediately following BTX-A injection and massage, above-knee casts were applied to the affected leg or legs. The initial casting was followed by three more casts, each 1 week apart. With each change, the foot was casted in maximum dorsiflexion to a maximum of 20 degrees. 3M Softcast was used as the cast material.

Bracing Protocol

At the end of the third week following BTX-A injection, Dennis Browne bar and corrective shoes (boots and bars) were fitted to the patient. The corrective shoes were set to the bar with external rotation between 55 and 65 degrees and the bar was bent to give dorsiflexion of 15 degrees. Custom-fitted knee-ankle-foot orthoses (KAFO) were required in a few patients due to intolerance of the boots and bars. The protocol for bracing used in this study was as follows: full time until weight bearing; during sleeping or resting once the child was weight bearing. Beyond 2 years of age, a trial of no bracing over 3 months was initiated. If any sign of recurrence was identified, then resumption of nighttime bracing occurred until 3 years of age. When patients were weight bearing, commercially available running shoes with specific features were used.

Example—Idiopathic Clubfoot Correction Outcomes in a Patient Group

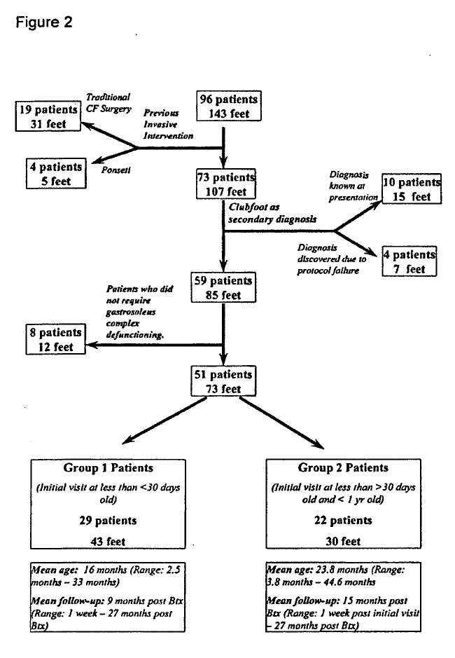

Fifty-one patients with 73 feet met the criteria for inclusion in the idiopathic clubfoot study, with 29 patients in group 1 and 22 patients in group 2. Mean age of patients for group 1 was 16 months (range 2.5-33) and average follow-up was 9 months after the BTX-A injection (range 1 week to 27 months). Mean age of patients for group 2 was 23.5 months (range 3.8-44.6) and average follow-up was 15 months after the BTX-A injection (range 1 week to 27 months) (FIG. 2).

All but one patient (one foot) who reached the point of hindfoot stall during the protocol of manipulations and castings had successful attenuation of the triceps surae complex using a single BTX-A injection. This patient presented at 4 months of age after previous attempts at manipulations and accelerated through the protocol. Poor maintenance of the hindfoot occurred after the BTX-A injection. Ultimately, the patient underwent a posterior release at 13 months of age.

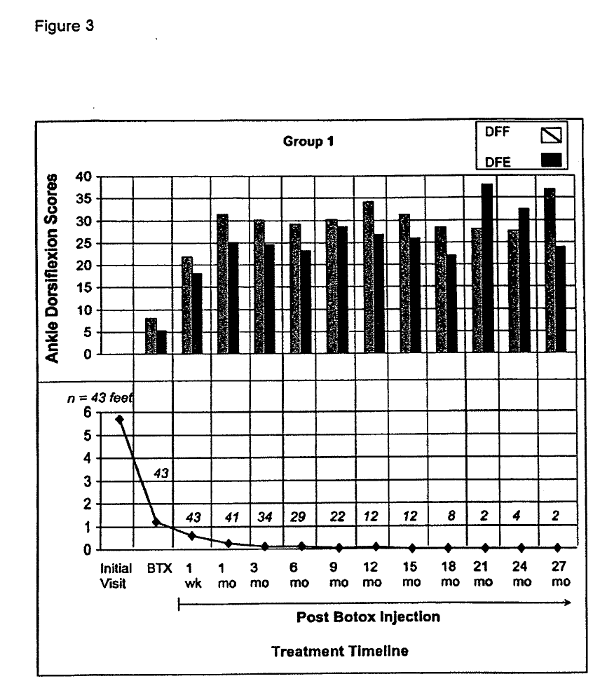

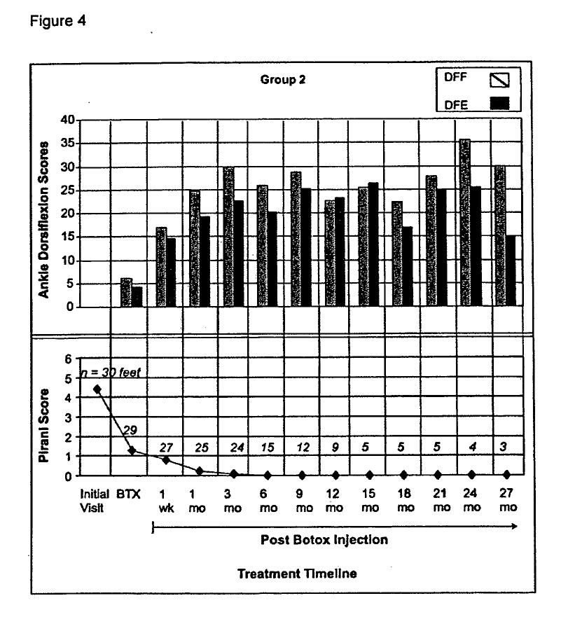

The mean initial Pirani score was 5.6±0.6 in group 1 and 4.5±1.9 in group 2 (P<0.05; power=0.897). This difference was accounted for by some group 2 patients who presented with partial corrections of the midfoot but hindfoot deformity still remained. By the day of BTX-A injection, the scores had decreased to a mean of 1.15±0.6 for group 1 and 1.5±1.1 for group 2 (P>0.05) (FIGS. 3 and 4).

Mean foot dorsiflexion scores are reported with the scores for the knee in flexion followed by the scores for the knee in full extension (Table 1; FIGS. 3 and 4). An increase in mean foot dorsiflexion scores was observed in the first week following BTX-A injection and further improvement occurred at the 1-month post-injection mark. These scores are reported in detail in Table 1. While measured dorsiflexion scores for the knee in flexion varied somewhat from visit to visit beyond this point, the mean remained in excess of 25 degrees for group 1 patients followed up to 12 months after the BTX-A injection and in the range of 20 degrees for group 2 patients. Measured dorsiflexion scores for the knee in full extension were maintained above 15 degrees for both groups beyond 12 months after the BTX-A injection.

TABLE 1
Improvement in Mean Foot Dorsiflexsion scores over time in idiopathic
clubfoot patients

| | Group 1 | | Group 2 | |
|---|---|---|---|---|
| | DFF | DFE | DFF | DFE |
| Day of BTX-A | 8.0° ± 11.6° | 5.3° ± 9.5° | 6.1° ± 9.7° | 4.2° ± 7.1° |
| 1 week post BTX-A | 21.8° ± 12.1° | 18.2° ± 11.8° | 16.9° ± 12.2° | 14.5° ± 10.5° |
| 1 month post BTX-A | 31.5° ± 11.8° | 25.2° ± 11.3° | 24.6° ± 9.7° | 19.4° ± 9.0° |

Scores are reported for knee in flexion (DFF) followed by knee in full extension (DFE).

No patients who received BTX-A experienced adverse reactions, side effects, or complications. Of the 50 patients who responded to the protocol, 9 patients lost some degree of dorsiflexion due to noncompliance with boots and bars, with fitting problems accounting for 2 cases. All these patients corrected with either a return to manipulations and casting alone (1 patient) or a combination of repeated BTX-A injection and further manipulations and castings (8 patients) (Table 2).

TABLE 2
Idiopathic clubfoot patients requiring a second intervention

| | Reason for | Time From | Age at | | Age as | Latest Scores | | |
|---|---|---|---|---|---|---|---|---|
| ID* | Relapse | BTX to Relapse | Relapse | Intervention | of Sept 03 | P | DFE | DFF |
| 1-3(l) | Compliance | 9 mo | 10 mo | Repeat BTX | 20 mo | 0 | 12 | 18 |
| | | | | and KAFO | | | | |
| 1-4(b) | Fitting | 1 mo | 3 mo | Repeat M&C | 26 mo | 0 | 32 | 34 |
| | ? | 9 mo | 11 mo | and convert to | | | | |
| | | | | KAFO | | | | |
| | | | | Repeat BTX, | | | | |
| | | | | M&C, KAFO | | | | |
| 1-21(b) | Compliance | 6 mo | 7 mo | Repeat M&C | 31 mo | 0 | 11 | 21 |
| 1-22(b) | Compliance | 5 mo | 6 mo | Repeat BTX, | 6.5 mo | 0 | 20 | 24 |
| | | | | M&C, and B&B | | | | |
| 1-24(b) | Compliance | 3 mo | 5 mo | Repeat M&C and | 30 mo | 0 | 12.5 | 17.5 |
| | Compliance | 4 mo | 6 mo | B&B | | | | |
| | Compliance | 16 mo | 18 mo | Repeat BTX. | | | | |
| | | | | M&C then KAFO | | | | |
| | | | | Switched to | | | | |
| | | | | articulated AFOs with | | | | |
| | | | | nighttime stretch strap | | | | |
| 2-11(r) | Compliance | 12 mo | 16 mo | Repeato BTX, | 47 mo | † | † | † |
| | | | | M&C, and B&B | | | | |
| 2-13(r) | Compliance | 9 mo | 19 mo | Repeat BTX, | 33 mo | 0 | 20 | 20 |
| | | | | M&C, and B&B | | | | |
| 2-17(r) | Fitting | 5 mo | 11 mo | Repeat BTX, | 16 mo | 0 | 20 | 20 |
| | | | | M&C, and B&B | | | | |
| 2-18(r) | Compliance | 2.5 mo | 9 mo | Repeat BTX, | 14 mo | 0 | 20 | 35 |
| | | | | M&C, and B&B | | | | |

*Patient assigned either as group 1(1) or group 2(2).
† Scores not available because patient not following up.
r, right clubfoot;
l, left clubfoot;
b, bilateral clubfeet;
M&C, manipulation and casting;
B&B, boots and bars;
BTX, botulinum Toxin A;
KAFO, knee-ankle-foot orthotic;
AFO, ankle-foot orthotic.

Example—Non-Idiopathic Clubfoot Correction Outcomes in a Patient Group.

In the non-idiopathic clubfoot population, the efficacy of Botox™ was defined as its capacity to correct clubfoot deformity. The primary outcome measure was based on the correction of the clubfoot deformity as measured by the amount of motion achieved by a subject which is the indicator for the degree of clubfoot correction (the degree of ankle dorsiflexion). The outcome measure is based on clubfoot treatment decisions, specifically, if the patient is able to fit into corrective bracing—all deformity has been sufficiently corrected such that bracing can be instituted (achieve ankle dorsiflexion of 10 degrees or greater) which is an indicator of correction of the clubfoot.

Non-idiopathic clubfoot patients include: three affected with meningomyelocole, six affected with arthrogryposis/anterior horn cell, one affected with developmental delay, one affected with trichorhinophalangeal syndrome, and three affected with migration abnormalities/cerebral palsy, for a total of fourteen patients. Thirteen of the fourteen of the non-idiopathic patients injected with Botox for their clubfoot received bracing (boots and bars or orthoses) indicating the achievement of correction.

While specific embodiments of the invention have been described and illustrated, such embodiments should be considered illustrative of the invention only and not as limiting the invention as construed in accordance with the accompanying claims.

Based on the foregoing, it will be appreciated that the present invention provides the following advances over the art:

1. A method of treating clubfoot in a patient, the method comprising; administering manipulation and casting therapy to an clubfoot-affected foot of a patient until hindfoot stall is achieved;

administering a medicament comprising a neuromuscular blocking agent to a triceps surae muscle complex adjoining said clubfoot-affected foot, and; applying a brace to said clubfoot-affected foot;

wherein said clubfoot-affected foot is held in a normal foot posture.

2. Said manipulation and casting therapy may comprise the Ponseti method.

3. Said medicament may comprise a botulinum toxin.

4. Said botulinum toxin may comprise botulinum toxin type A.

5. Said brace may comprise a Denis-Browne boot and brace.

6. Said clubfoot may be idiopathic or non-idiopathic.

7. Said non-idiopathic clubfoot may coexist with myelomenginocoele, arthrgryposis, migration abnormalities of the brain, cerebral palsy, positional deformity, neurological disorders, spina bifida, trichorhinophalangeal syndrome or a genetic syndrome resulting in a clubfoot deformity.

8. A method of diagnosing hindfoot stall in a patient receiving treatment for clubfoot, the method comprising;

acquiring a first Pirani score for a clubfoot affected foot of a patient; acquiring a second Pirani score for said clubfoot-affected food of a patient at a later time;

comparing said second Pirani score with said first Pirani score, and;

determining a magnitude of change,

wherein said magnitude of change less than a cut-off value indicates hindfoot stall in a clubfoot-affected foot of said patient.

9. Said cutoff value may comprise an ankle dorsiflexion range from about −5 degrees to about +5 degrees.

10. Said clubfoot may be idiopathic or non-idiopathic.

11. Said non-idiopathic clubfoot may coexist with myelomenginocoele, arthrgryposis, migration abnormalities of the brain, cerebral palsy, positional deformity, neurological disorders, spina bifida, trichorhinophalangeal syndrome or a genetic syndrome resulting in a clubfoot deformity.

12. A method of treating hindfoot stall in a patient receiving treatment for clubfoot, the method comprising;

administering to a clubfoot-affected foot of a patient diagnosed with hindfoot stall a medicament comprising botulinum toxin to a triceps surae muscle complex adjoining said clubfoot-affected foot;

applying a brace to said clubfoot-affected foot, and;

wherein said clubfoot-affected foot is held in a normal posture.

13. Said medicament may comprise a neuromuscular blocking agent.

14. Said neuromuscular blocking agent may comprise a botulinum toxin.

15. Said botulinum toxin may comprise botulinum toxin type A.

16. Said brace may comprise a Denis-Browne boot and brace.

17. Said clubfoot may be idiopathic or non-idiopathic.

18. Said non-idiopathic clubfoot may coexist with myelomenginocoele, arthrgryposis, migration abnormalities of the brain, cerebral palsy, positional deformity, neurological disorders, spina bifida, trichorhinophalangeal syndrome or a genetic syndrome resulting in a clubfoot deformity.

What is claimed is:

1. A method of treating clubfoot in a patient, the method comprising;
administering manipulation and casting therapy to an clubfoot-affected foot of a patient until hindfoot stall is achieved;
administering a medicament comprising a neuromuscular blocking agent to a triceps surae muscle complex adjoining said clubfoot-affected foot, and;
applying a brace to said clubfoot-affected foot;
wherein said clubfoot-affected foot is held in a normal foot posture.
